(19) United States
(12) Patent Application Publication
(10) Pub. No.: US 2026/0360499 A1
(43) Pub. Date: Nov. 12, 2026

(54) Systems and methods for dispensing a prescription medication

(71) Applicant: SMARTRX PTE. LTD, Singapore (SG)

(72) Inventors: Ping Heng Tong, Singapore (SG); Whee Keong Bennet Lee, Singapore (SG); Chen Hao Tong, Singapore (SG)

(21) Appl. No.: 19/137,165

(22) PCT Filed: Dec. 15, 2023
(86) PCT No.: PCT/SG2023/050838

§ 371 Date: Jun. 9, 2025

(30) Foreign Application Priority Data
Dec. 16, 2022 (SG) 10202260480W

(51) Int. Cl. G16H 20/13; G16H 30/40; G16H 40/67

(52) U.S. Cl. CPC G16H 20/13; G16H 30/40; G16H 40/67

(57) ABSTRACT

A system and method for dispensing a prescription medication are provided. The system includes a user identification module configured to identify a user; a prescription retrieval module configured to retrieve a prescription associated with the identified user from a server that is remotely connected to the system; a labelling module configured to label the prescribed prescription medication based on the retrieved prescription; and a dispensing module configured to dispense the labelled prescribed prescription medication.

### FIELD OF INVENTION

The present invention relates broadly, but not exclusively, to systems for dispensing a medication and, more particularly, to systems for dispensing a prescription medication and methods thereof.

### BACKGROUND OF THE DISCLOSURE

Typically, there are three main classifications and levels of access controls for medication products, namely prescription only medicines (POM) or prescription medication, pharmacy only medicines (P-med) and general sales list (GSL).

POM or prescription medication is a therapeutic product that can only be obtained from a doctor or a dentist, or from a pharmacist with a prescription from a doctor or a dentist. The condition to be treated needs to be diagnosed and treated by a doctor, said condition can also have serious side effects which require a doctor's monitoring or follow up.

P-med is a therapeutic product that can be obtained from a pharmacist at a retail pharmacy. The condition to be treated is self-limiting and can be assessed and treated by pharmacists.

GSL is a therapeutic product that can be freely obtained from any retailer. It can be used safely by the public without medical supervision and is intended for short term self-treatment only.

A typical P-med and prescription medication dispensing process involves significant number of manhours. Conventionally, P-med and prescription medication dispensing processes used in hospitals, clinics and pharmacies involve stringent manual checks and manual verifications. Prescription medication dispensing processes further involve manual labelling of the medication by medical professionals before the medication can be dispensed to the patient. With increasing need for healthcare services in many countries due to various reasons such as an aging population, there is an increased need for prescription medication dispensing services. However, increasing manpower shortage issues in healthcare industries poses a problem to the increased need for prescription medication dispensing services.

Further, a typical patient in a remote area such as a dormitory, or a village such as a municipal may have limited or no access to prescription medication dispensing services as there is seldom any medical professional present in the remote area or the village to perform the manual checks, manual verifications and manual labelling of the medication.

A need therefore exists to provide a system for dispensing a prescription medication that seeks to address at least some of the above problems. Furthermore, other desirable features and characteristics will become apparent from the subsequent detailed description and the appended claims, taken in conjunction with the accompanying drawings and this background of the disclosure.

### SUMMARY

According to a first aspect, there is provided a system for dispensing a prescription medication, comprising: a user identification module configured to identify a user; a prescription retrieval module configured to retrieve a prescription associated with the identified user from a server that is remotely connected to the system; a labelling module configured to label the prescribed prescription medication based on the retrieved prescription; and a dispensing module configured to dispense the labelled prescribed prescription medication.

According to a second aspect, there is provided a method for dispensing a prescription medication, comprising: identifying a user; establishing a connection between a system for dispensing the prescription medication and a remote server; retrieving a prescription associated with the identified user from the remote server; labelling the prescribed prescription medication based on the retrieved prescription; and dispensing the labelled prescribed prescription medication.

### BRIEF DESCRIPTION OF THE DRAWINGS

Embodiments and implementations are provided by way of example only, and will be better understood and readily apparent to one of ordinary skill in the art from the following written description, read in conjunction with the drawings, in which:

FIG. 1A is a block diagram of a system for dispensing a prescription medication, according to an example embodiment.

FIG. 1B is a block diagram of the system for dispensing the prescription medication of FIG. 1A when used for general sales list medication, pharmacy only medicines and prescription medication, according to an example embodiment.

FIG. 2 is a schematic representation of the system for dispensing the prescription medication of FIG. 1A, according to an example embodiment.

FIG. 3 is a schematic representation of a vital signs data collection booth, according to an example embodiment.

FIG. 4 is a schematic representation of a combined system comprising the system for dispensing the prescription medication of FIG. 2 and the vital signs data collection booth of FIG. 3, according to an example embodiment.

FIG. 5 shows the system for dispensing the prescription medication of FIG. 2, the vital signs data collection booth of FIG. 3 and the combined system of FIG. 4 deployed for use in healthcare institutions, malls, municipalities and remote areas, according to an example embodiment.

FIG. 6 shows the combined system of FIG. 4 deployed for use in a hospital, according to an example embodiment.

FIG. 7 shows the system for dispensing the prescription medication of FIG. 2 deployed for use in malls and the combined system of FIG. 4 deployed for use in dormitories and municipalities, according to an example embodiment.

FIG. 8 is a flowchart illustrating a method for dispensing a prescription medication, according to an example embodiment.

Skilled artisans will appreciate that elements in the figures are illustrated for simplicity and clarity and have not necessarily been depicted to scale.

### DETAILED DESCRIPTION

Embodiments will be described, by way of example only, with reference to the drawings. Like reference numerals and characters in the drawings refer to like elements or equivalents.

As mentioned above, a typical prescription medication dispensing process involves significant number of manhours as it involves stringent manual checks, manual verifications and manual labelling of a prescription medication by medical professionals before the prescription medication can be dispensed to a patient. Further, a typical patient in a remote area such as a dormitory or a village such as a municipal may have limited or no access to prescription medication dispensing services as there is seldom any medical professional present in the remote area or the village to perform the manual checks, manual verifications and manual labelling of the prescription medication.

Embodiments of the invention advantageously provide a system for dispensing the prescription medication with a reduced number of manhours, hence improves efficiency. Beneficially, the system for dispensing the prescription medication can also be deployed for use in the remote areas and the villages, hence providing patients in these locations with access to prescription medication dispensing services. Personnel who can benefit from the system may include medical personnel providing medical services such as teleconsultation services and remote dispensing of medication, users such as patients purchasing medication and/or using the teleconsultation services, restocking personnel providing restocking services to restock medication, and system administrators managing a system software.

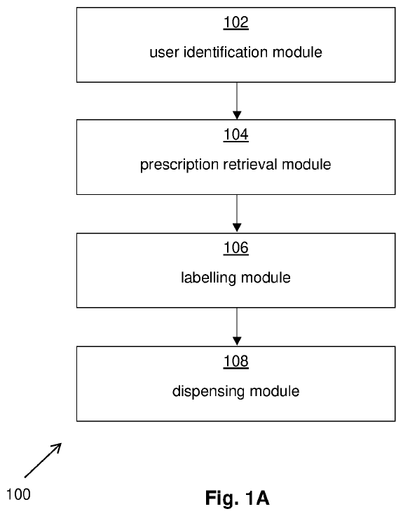

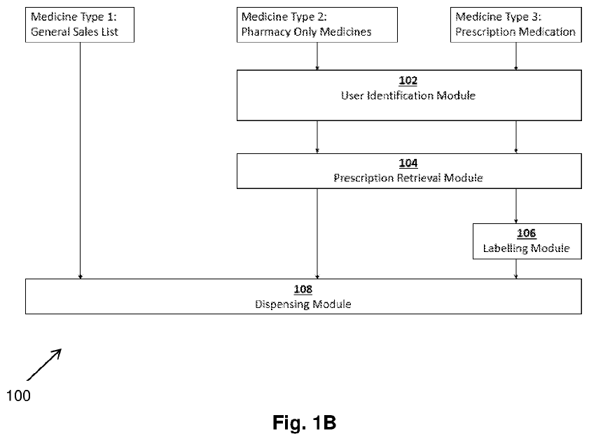

FIG. 1A is a block diagram 100 of a system for dispensing a prescription medication, according to an example embodiment. The system for dispensing the prescription medication comprises a user identification module 102 configured to identify a user. The system further comprises a prescription retrieval module 104 configured to retrieve a prescription associated with the identified user from a server that is remotely connected to the system. The system also comprises a labelling module 106 configured to label the prescribed prescription medication based on the retrieved prescription. Further, the system comprises a dispensing module 108 configured to dispense the labelled prescribed prescription medication. The user can be a patient.

It would be appreciated that the system for dispensing the prescription medication can also be used for general sales list (GSL) medication, pharmacy only medicines (P-med), medical devices, dental products and traditional medicine products. GSL medication can typically be purchased by users without supervision of medical personnel. Purchase of GSL medication may be performed in physical and manned locations such as retail pharmacies. Being able to dispense GSL medication from the system for dispensing the prescription medication, which can be an automated system, streamlines the payment process and collection process, thereby increasing accessibility of basic healthcare and enhancing efficiency of purchasing GSL medication.

In the event of a global pandemic, workload of healthcare professionals may be increased. Personal health risks faced by the healthcare professionals and patients may be increased as well. Being able to dispense medication from the system for dispensing the prescription medication, which can be a remote dispensing system, reduces risks of virus transmission from face-to-face or physical interactions. At the same time, quality of healthcare services provided is maintained or improved. Further, as would be explained in more details later, provisions for add on modules such as a vital signs collection module can allow the user to collect his/her own vital signs independently. Remote teleconsultation may also be performed. These capabilities reduce the need for users such as patients to visit hospitals which in turn frees up healthcare resources and saves time for the users.

FIG. 1B is a block diagram 100 of the system for dispensing the prescription medication of FIG. 1A when used for GSL medication, P-med and prescription medication, according to an example embodiment. As shown in FIG. 1B, when the system is used for GSL medication, the dispensing module 108 may be activated. When the system is used for P-med, the user identification module 102, the prescription retrieval module 104 and the dispensing module 108 may be activated. When the system is used for prescription medication, the user identification module 102, the prescription retrieval module 104, the labelling module 106 and the dispensing module 108 may be activated.

According to one embodiment, the user identification module 102 may comprise a data capturing module configured to capture a user data for identifying the user. As a non-limiting example, the data capturing module may comprise a user biometrics recognition system such as a face recognition system, an iris recognition system and/or a fingerprint recognition system. Accordingly, the data capturing module may comprise a camera for capturing biometrics of the user such as facial features and/or unique patterns in an iris of the user, and/or a fingerprint scanner for capturing unique patterns in a fingerprint of the user. The camera may also capture user details in government records such as from government linked applications installed in a mobile device of the user. The camera may be configured to capture static images and/or videos. It would be appreciated that the camera may also be used for teleconsultation purposes. The system for dispensing the prescription medication may also comprise microphone(s) and/or speaker(s) which may facilitate two-way communication between the user and the medical personnel for teleconsultation purposes. The data capturing module may also comprise a user identification card recognition system and/or a user interface that allows the user to manually input his/her personal particulars. Accordingly, the data capturing module may comprise an identification card scanner for capturing details of an identification card of the user, a keypad and screen, and/or a touchscreen for the user to manually input his/her personal particulars. The user identification module 102 may also comprise a processor for processing the captured user data to identify the user.

In an implementation, the server may be further connected to a pre-determined remote healthcare system. The pre-determined remote healthcare system may transmit the prescription to the server. The prescription retrieval module 104 may comprise an Application Programming Interface (API) that can connect the system for dispensing the prescription medication to the pre-determined remote healthcare system via the server. The pre-determined remote healthcare system can be a healthcare services software application system of a hospital or a clinic and/or a healthcare distributed workload software application system of the hospital or the clinic. Doctors and/or pharmacists authorized to use the system for dispensing the prescription medication can connect to the system for dispensing the prescription medication by accessing the pre-determined remote healthcare system.

The system for dispensing the prescription medication may comprise a lift system such as a robotic lift system that can move a container for containing the medication. The system for dispensing the prescription medication may also comprise a robotic extension such as a robotic arm in an axis such as a XY axis to retrieve the medication from a specific lane and transfer the medication to the modules such as the dispensing module. The system for dispensing the prescription medication may also comprise a backend API or a software configured to tag or detect a category or a type of the medication, hence identify an access control level of the medication. The backend API or the software may be configured to compute an order of retrieving and/or dispensing the medication. Efficiency may be achieved in this manner. The speed of dispensing the medication may be adjusted based on predetermined requirements. It would be appreciated that different categories or types of the medication can be dispensed using the same system for dispensing the prescription medication.

The medication identified as a GSL medication can be transferred to the dispensing module 108 directly. In a scenario that the medication is identified as a P-med, users may be prompted to initiate a remote consultation with medical personnel such as a pharmacist. After the user has identified himself/herself using the user identification module 102, the medical personnel may remotely control the system for dispensing the prescription medication to retrieve a specific medication. The medical personnel may be able to watch, for example using a video system, the retrieval process and verify the medication before the medication is transferred to the dispensing module 108.

In a scenario that the medication is identified as a prescription medication, users may be prompted to initiate a remote consultation with a doctor. The doctor can prescribe the prescription medication and send the prescription to the server. The doctor may remotely control the system for dispensing the prescription medication to retrieve the prescription medication. The system for dispensing the prescription medication may retrieve the prescription medication and transfer the prescription medication to the labelling module 106. Thereafter, the prescription medication may be transferred to the dispensing module 108. As mentioned above, the pre-determined remote healthcare system may transmit the prescription to the server. In this scenario, the user may not be prompted to initiate the remote consultation with the doctor. The user may collect the prescribed prescription medication at any time or at a pre-determined time. The system for dispensing the prescription medication may initiate the user identification module. A two-factor authentication process may also be initiated. The two-factor authentication process may include an internal security authentication system and/or an external security authentication system to enhance accuracy of identifying the user, hence improves security.

According to one embodiment, the labelling module 106 may comprise a printing module configured to print a label comprising a user name of the user, a user identification number of the user, a lot number or a batch number of the medication, an expiry date of the medication and a medication instruction based on the prescription. The medication instruction may include dosage, intake frequency, cautionary information and/or advice of the doctor. The labelling module 106 may also comprise an affixing module configured to affix the printed label to a container containing the prescribed prescription medication. The labelling module 106 may further comprise a processor incorporated with an artificial intelligence (AI) technology. The processor may be configured to process an image of the container to identify a position and/or an orientation of the container. A preferred area on the container for affixing the printed label may be computed. The printed label may be affixed at the preferred area on the container. The container can be a rigid container such as a box or a bottle, or a flexible packaging such as a plastic packaging. Further, the labelling module 106 may comprise a robotic arm and/or a conveyor system for transferring the printed label to the container containing the prescribed prescription medication. The robotic arm or another robotic arm may be used to affix the printed label to the container.

The system for dispensing the prescription medication may further comprise a verification module configured to facilitate verification of the labelled prescribed prescription medication before the dispensing module 108 dispenses the labelled prescribed prescription medication.

The verification module may comprise an image capturing module configured to provide static images and/or real-time video streaming of the labelling module and/or medication to be dispensed to medical personnel. The medical personnel may connect to the system for dispensing the prescription medication by logging in to a healthcare system that can be remotely connected to the system for dispensing the prescription medication. The medical personnel may be a doctor or a pharmacist. The images and/or the videos obtained by the image capturing module may be used for troubleshooting and future verification purposes.

Further verification of the labelled prescribed prescription medication may be performed before the dispensing module 108 dispenses the labelled prescribed prescription medication. For example, the prescription medication or printed label may comprise a radio frequency identification (RFID) tag, a barcode or a quick response code (QR code) that may be scanned and verified before the dispensing module 108 dispenses the labelled prescribed prescription medication.

The system for dispensing the prescription medication may further comprise a payment module configured to facilitate the user in making payment for the prescribed prescription medication. The payment module may comprise a processor configured to calculate a payment amount based on the prescribed prescription medication. The payment module may be configured to accept payment via multiple channels including but not limited to credit cards, debit cards, or payment using mobile devices. The medication may be retrieved and dispensed after payment has been successfully made.

The dispensing module 108 may comprise the robotic arm and/or the conveyor system of the labelling module 106 and/or another robotic arm and/or another conveyor system to transfer the labelled prescribed prescription medication to an outlet for collection by the user.

The outlet may comprise a physical barrier which can restrict access to unauthorized personnel. Advantageously, the physical barrier provides better control and enhanced safety for dispensing the prescription medication and the P-med. After confirming that a correct medication as well as a correct quantity of each medication has been dispensed, the medical personnel such as the pharmacist or the doctor may be required to transmit a signal to the system for dispensing the prescription medication to deactivate a lock that may hold the physical barrier in place so that after the lock is deactivated, the user can collect the medication. This step may act as a checkpoint to ensure that users can only gain access to the types and quantities of medicine prescribed by the medical personnel, thus enhancing safety. The outlet may also comprise a safety system configured to prevent a user's hand from being trapped by the physical barrier when collecting the medication. The system for dispensing the prescription medication may further comprise an audio or a visual reminder system configured to remind the user to collect the medication. The physical barrier can be triggered to reopen if the user requires more time to collect the medication.

A medication clearing system configured to clear an incorrect medication or an uncollected medication may be installed at the outlet. The medication clearing system can transfer the incorrect medication or the uncollected medication to a compartment which may be inaccessible to public. Advantageously, in a scenario that an incorrect medication is transferred to the outlet or the user fails to collect the medication, the incorrect medication or the uncollected medication can be transferred to the compartment to prevent subsequent users from accessing the medication. The medication clearing system may be connected to the verification module. When the pharmacist or the doctor using the verification module spots incorrect medication being dispensed or uncollected medication left behind in the outlet, they can activate the medication clearing system.

The system for dispensing the prescription medication may comprise a failure alert system configured to send a failure signal to the server if dispensing fails during a dispensing process. The failure alert system may also determine if the system for dispensing the prescription medication can continue to serve a next user. If it is determined that the system for dispensing the prescription medication cannot serve the next user, the system for dispensing the prescription medication may automatically enter into a maintenance mode. The failure alert system may advise users on remedial action(s) available.

The user may also collect a GSL or an over-the-counter (OTC) medication from the system. As would be appreciated, the labelling module may not be required as there is no requirement to label the GSL medication and the OTC medication. The system may comprise a medication identification module configured to identify whether the medication to be dispensed is the prescribed prescription medication or the GSL medication or the OTC medication.

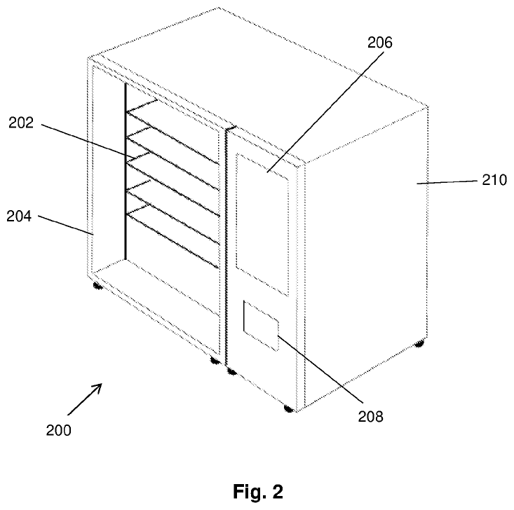

FIG. 2 is a schematic representation 200 of the system for dispensing the prescription medication of FIG. 1A, according to an example embodiment. As shown in FIG. 2, the system for dispensing the prescription medication may comprise a rack 202 for placing the prescription medication and a door 204 to prevent unauthorized personnel from accessing the prescription medication. The door 204 may have a clear glass to allow visibility of the prescription medication placed on the rack 202. The door 204 may also have an opaque finish. A metal material may also be used for the door 204. The door 204 may also be a large digital panel or may be mounted with a digital screen. As mentioned above, the system for dispensing the prescription medication may also comprise the data capturing module 206 and the outlet 208 for collection by the user. As shown in FIG. 2, the prescription retrieval module 104, the labelling module 106, the verification module and the dispensing module 108 may be disposed in an enclosure 210 wherein the prescription retrieval module 104, the labelling module 106, the verification module and the dispensing module 108 may not be visible and/or accessible to the user. Beneficially, non-authorized persons cannot meddle with the system, hence access to the medication is strictly controlled.

The system for dispensing the prescription medication may comprise a temperature control system, a humidity control system and/or a lighting control system. The temperature control system, the humidity control system and the lighting control system may be configured based on requirements for storage of the GSL medication, the P-med and/or the prescription medication. The temperature control system, the humidity control system and the lighting control system may comprise sensor(s) to sense an environmental condition at the rack 202. Data of the environmental condition at the rack 202 may be collected and recorded. Data log(s) may be generated. The recorded data and generated data log(s) may be used for audit purposes. Further, personnel managing the system for dispensing the prescription medication may be alerted if the collected data is out of a predetermined range corresponding to the requirements for storage of the medication.

The system for dispensing the prescription medication may further comprise a restocking module configured to detect a quantity of the prescription medication. The restocking module may also be configured to track the batch or lot number of the medication and the expiry date of the medication. The restocking module may be further configured to transmit a low inventory signal and/or a medication expiring signal to the server in response to detecting that the quantity of the prescription medication is less than a predetermined quantity and/or the medication is close to the expiry date, respectively. The restocking module may also be configured to detect a quantity of the P-med and GSL medication. The restocking module may also be configured to transmit the low inventory signal to the server in response to detecting that the quantity of the P-med and GSL medication is less than the predetermined quantity. Beneficially, the restocking module reduces manhours required to perform periodic manual checks on the quantity of the prescription medication, the P-med and the GSL medication.

The restocking module can also be configured to reduce human error during a medication restocking process, and ensure that the medication are correctly loaded at their respective lanes on the rack 202. Each type of medication or each of the medication may be tagged or labelled with a corresponding unique code such as a QR code, a barcode or a RFID tag. The restocking module may comprise a scanner configured to scan and read the unique code. The restocking module may also comprise a backend API configured to process data associated with the medication such as a lot number, an expiry date, the type of medication, a quantity and/or a lane number in which the medication is placed. The restocking module may further comprise a guidance system for guiding a restocking personnel to correctly restock the medication at the correct lanes on the rack 202. The guidance system may comprise an audio guidance system, a visual guidance system such as LED light(s), and/or a control system configured to control the audio guidance system and/or the visual guidance system. For example, audio instructions may be provided to guide the restocking personnel to restock the medication at the correct lanes on the rack 202. Steady lights or blinking lights may also be provided at each lane on the rack 202 to guide the restocking personnel to restock the medication at a particular lane. The guidance system may also comprise a RFID system to guide the restocking personnel to restock the correct medication, correct quantity of the medication and to the correct lane and rack.

The restocking module may comprise a safety system configured to prevent unexpected movement of parts of the system for dispensing the prescription medication during the restocking process. This provides improved safety for the restocking personnel. Further, the restocking module may be configured to prevent unauthorized access to the medication. For example, identity of the restocking personnel may be verified before the door 204 can be opened for restocking purposes.

It would be appreciated that operations such as changing a rack planogram, opening and/or closing the door 204, refreshing or rebooting the systems of the system for dispensing the prescription medication, updating information such as the quantity of the medication, a price of the medication, display images of the medication and a description of the medication, can be performed locally or remotely. The operations can also be performed in real-time or based on a predetermined schedule.

The system for dispensing the prescription medication may further comprise a vital signs collection module configured to collect one or more vital signs data of the user. The vital signs collection module may be device agnostic. The one or more vital signs data may comprise a body temperature reading, a blood pressure reading, a heart rate reading, a blood oxygen level reading, a height measurement reading and/or a weight measurement reading. The height measurement reading and the weight measurement reading may be used to calculate a Body Mass Index (BMI) of the user. Accordingly, the vital signs collection module may comprise vital signs collection devices such as a thermometer for obtaining the body temperature reading of the user, a blood pressure (BP) meter for obtaining the blood pressure reading of the user, a Holter monitor or a heart rate monitor for obtaining the heart rate reading of the user, an oximeter for obtaining the blood oxygen level reading of the user, a stadiometer for obtaining the height measurement reading of the user and/or a weighing scale for obtaining the weight measurement reading of the user. Other vital signs collection devices may be included.

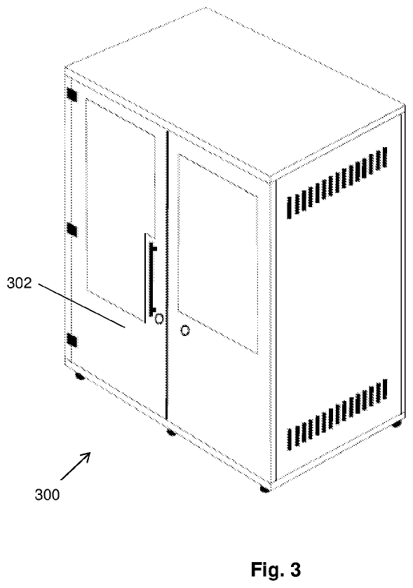

FIG. 3 is a schematic representation of a vital signs data collection booth 300, according to an example embodiment. The vital signs collection module may be disposed in the vital signs data collection booth 300. As shown in FIG. 3, the vital signs data collection booth 300 may be designed such that the user can physically walk into the booth 300. The booth 300 may also have doors 302 to allow privacy of the user. The doors 302 may also provide access control such that, for example, only a pre-registered user and/or a user seeking medical services may be allowed entry into the booth 300.

The booth 300 may be equipped with a sanitizing system such as a UV sanitizing unit for sanitizing the internal space of the booth 300. The sanitizing system may be activated according to a pre-determined setting. As a non-limiting example, the pre-determined setting can be a fixed duration such that the booth 300 is sanitized at a fixed frequency. The pre-determined setting can also correspond to a detection of the user leaving the booth 300. For example, a detection unit such as a motion sensor may be installed in the booth 300 to detect a presence of the user in the booth 300. After the user leaves the booth 300, the detection unit will trigger the activation of the sanitizing system. In this manner, advantageously, a risk of germs or viruses spreading between the user and a next user is reduced. Further, the booth 300 may be equipped with an air purifying unit for purifying the air in the booth 300. The air purifying unit can be activated according to the pre-determined setting as described above.

The booth 300 may comprise another user identification module configured to identify the user. The user identification module can be one that is similar to that described above. The booth 300 may also comprise a user interface such as a screen, for example an interactive screen, and an audio system. The user interface can guide the user with visual images and/or audio instructions on using the vital signs collection devices. The user may be guided using audio recordings, visual recordings and/or a remote consultation with the medical personnel. Beneficially, the user is guided to use the vital signs collection devices with ease. The booth 300 may further comprise a real time correction engine to provide more guidance to the user. Beneficially, if the user faces difficulties using the vital signs collection devices or collects erroneous data, the real time correction engine can rectify the user's mistakes every step along the way.

According to one embodiment, the server is further connected to the vital signs collection module. The user interface of the booth 300 may further guide the user to manually upload the one or more vital signs data obtained to the server. The vital signs collection devices may also be connected to the server via wireless or cellular communications gateway. As such, beneficially, the one or more vital signs data obtained is transmitted to the server without human intervention. The vital signs collection devices may also be equipped with Bluetooth functions. Beneficially, the booth 300 allows the user to request for a remote consultation session with the doctor who is authorized to use the system for dispensing the prescription medication. The doctor can interact with the user and diagnose an ailment of the user based on the one or more vital signs data that has been transmitted to the server. In other words, the doctor can provide a remote consultation to the user by accessing the pre-determined remote healthcare system which is connected to the server. Advantageously, the clinical diagnosis made by the doctor is more accurate.

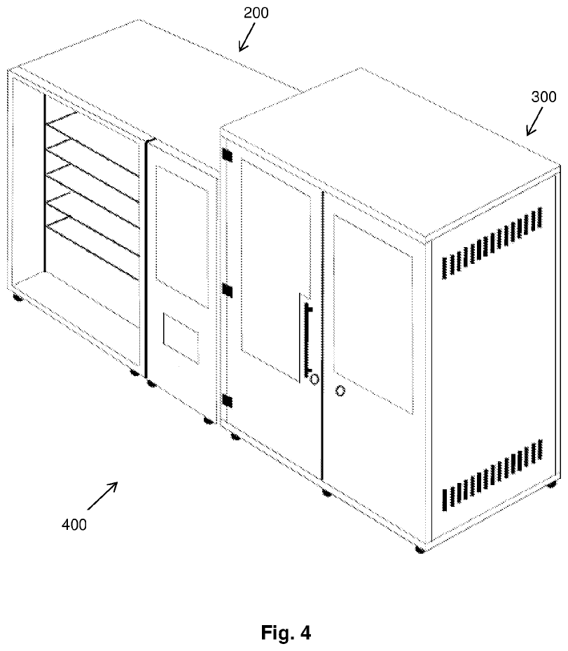

FIG. 4 is a schematic representation of a combined system 400 comprising the system for dispensing the prescription medication of FIG. 2 and the vital signs data collection booth 300 of FIG. 3, according to an example embodiment. The booth 300 may be detachably coupled to the system for dispensing the prescription medication. The booth 300 and the system for dispensing the prescription medication may also be disposed separately. It would be appreciated that signals and data can be transmitted between the booth 300 and the system for dispensing the prescription medication. As mentioned above, the booth 300 can allow the user to request for the remote consultation session with the doctor who is authorized to use the system for dispensing the prescription medication. The doctor can also receive an updated medication inventory list of the medication available in the system. The updated medication inventory list and/or a location of the system for dispensing the prescription medication may be made available to the public or the user. Upon diagnosing the ailment of the user via the remote consultation session, the doctor can prescribe the prescription based on the ailment and/or the updated medication inventory list. The prescription can then be uploaded to the server. The prescription retrieval module 104 can retrieve the prescription from the server thereafter. Beneficially, the user is able to collect his/her labelled prescribed prescription medication shortly after the remote consultation session ends. This improves the quality of healthcare services provided to the users.

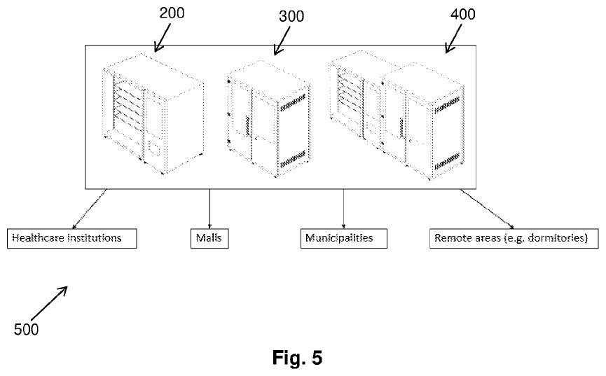

FIG. 5 500 shows the system for dispensing the prescription medication of FIG. 2, the vital signs data collection booth of FIG. 3 and the combined system of FIG. 4 deployed for use in healthcare institutions such as hospitals and polyclinics, malls, municipalities and remote areas such as dormitories, according to an example embodiment. The system for dispensing the prescription medication, the vital signs data collection booth or the combined system can be deployed for use in each of the areas based on needs of the users in the area.

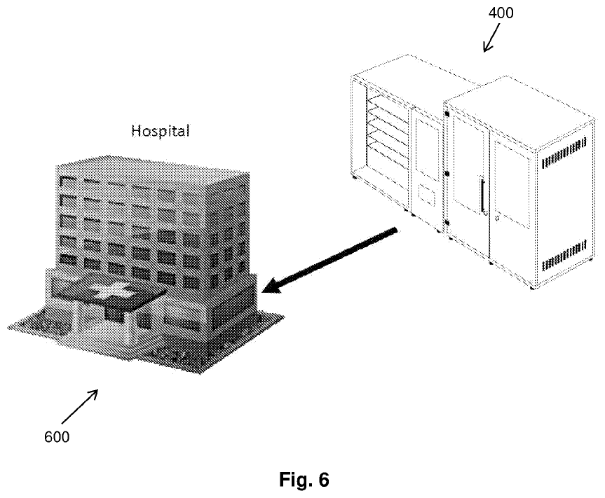

The system for dispensing the prescription medication, the vital signs data collection booth 300 and/or the combined system 400 may be deployed within a premises of a healthcare institution to provide an additional means for users to obtain their medication. Advantageously, workload in a pharmacy of the healthcare institution can be reduced. Hence, waiting times of the users or the patients may be reduced too. Further, as the users or the patients can collect his/her vital signs data by himself/herself, workload of the medical personnel in the healthcare institution can be reduced. FIG. 6 600 shows the combined system 400 of FIG. 4 deployed for use in a healthcare institution such as a hospital, according to an example embodiment.

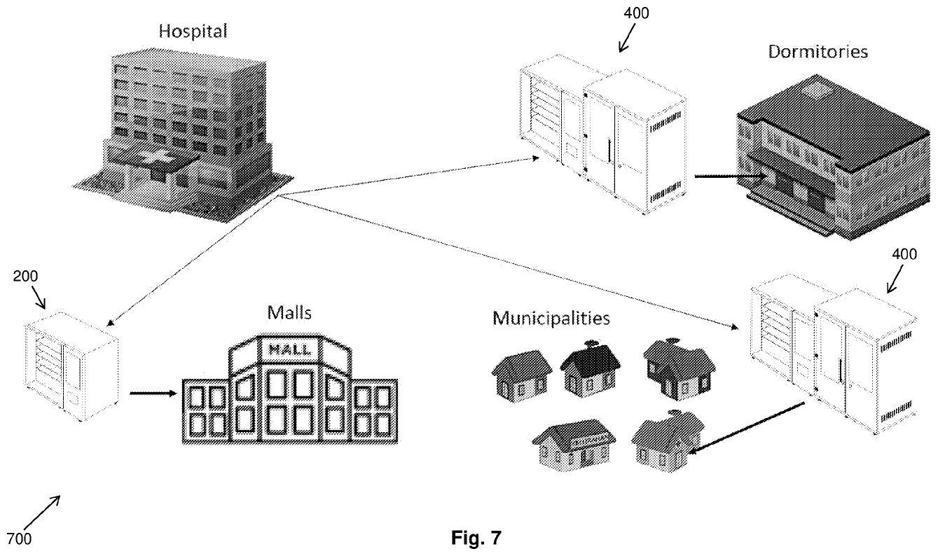

FIG. 7 700 shows the system for dispensing the prescription medication of FIG. 2 deployed for use in malls and the combined system 400 of FIG. 4 deployed for use in dormitories and municipalities, according to an example embodiment. This allows healthcare institutions to extend their services outside the boundaries of their physical institutions and bring quality care to relatively remote areas.

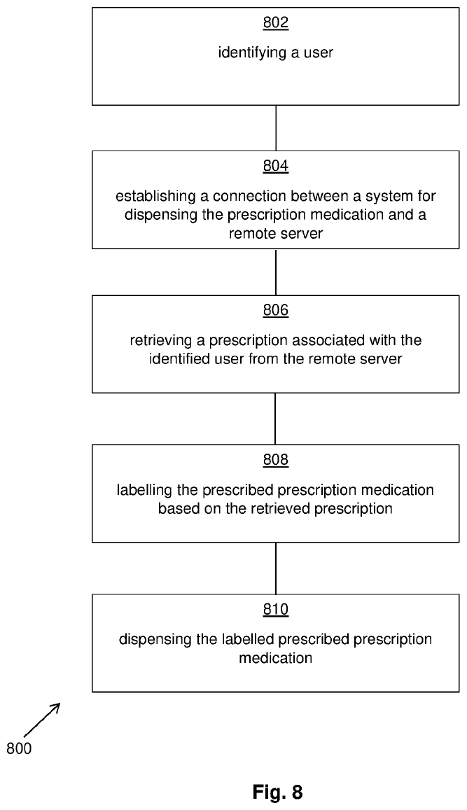

FIG. 8 is a flowchart 800 illustrating a method for dispensing a prescription medication, according to an example embodiment. At step 802, a user is identified using a user identification module. At step 804, a connection between a system for dispensing the prescription medication and a remote server is established. At step 806, a prescription associated with the identified user from the remote server is retrieved using a prescription retrieval module. At step 808, the prescribed prescription medication based on the retrieved prescription is labelled using a labelling module. At step 810, the labelled prescribed prescription medication is dispensed using a dispensing module.

According to one embodiment, identifying the user may comprise capturing a user data for identifying the user. Labelling the prescribed prescription medication may comprise printing a label comprising a user name of the user and a medication instruction based on the prescription. Labelling the prescribed prescription medication may also comprise affixing the printed label to a container containing the prescribed prescription medication.

The method for dispensing the prescription medication may further comprise verifying, using a verification module, the labelled prescribed prescription medication before dispensing the labelled prescribed prescription medication. According to one embodiment, verifying the labelled prescribed prescription medication may comprise providing, using an image capturing module, real-time video streaming of the labelling module to a medical personnel. The method for dispensing the prescription medication may further comprise collecting, using a vital signs collection module, one or more vital signs data of the user. The one or more vital signs data may comprise a body temperature reading, a blood pressure reading, a blood oxygen level reading, a height measurement reading and/or a weight measurement reading.

The method for dispensing the prescription medication may further comprise establishing a connection between the remote server and a pre-determined remote healthcare system. The pre-determined remote healthcare system may transmit the prescription to the remote server. According to one embodiment, the method for dispensing the prescription medication may further comprise detecting, using a restocking module, a quantity of the prescription medication. Further, the method for dispensing the prescription medication may comprise in response to detecting that the quantity of the prescription medication is less than a predetermined quantity, transmitting using the restocking module, a low inventory signal to the remote server.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive.

1. A system for dispensing a prescription medication, comprising:
a user identification module configured to identify a user;
a prescription retrieval module configured to retrieve a prescription associated with the identified user from a server that is remotely connected to the system;
a labelling module configured to label the prescribed prescription medication based on the retrieved prescription;
a verification module configured to facilitate verification of the labelled prescribed prescription medication, wherein the verification module comprises an image capturing module configured to provide real-time video streaming of the labelling module to a medical personnel for the medical personnel to verify the labelled prescribed prescription medication; and
a dispensing module configured to dispense the verified prescribed prescription medication.

2. The system of claim 1, wherein the user identification module comprises a data capturing module configured to capture a user data for identifying the user.

3. The system of claim 1, wherein the labelling module comprises:
a printing module configured to print a label comprising a user name of the user and a medication instruction based on the prescription; and
an affixing module configured to affix the printed label to a container containing the prescribed prescription medication.

4. The system of claim 1, further comprising a vital signs collection module configured to collect one or more vital signs data of the user.

5. The system of claim 4, wherein the vital signs collection module comprises vital signs collection devices, and wherein the user is guided to use the vital signs collection devices via a remote consultation with the medical personnel.

6. The system of claim 4, wherein the one or more vital signs data comprises a body temperature reading, a blood pressure reading, a blood oxygen level reading, a height measurement reading and/or a weight measurement reading.

7. The system of claim 4, wherein the server is further connected to the vital signs collection module.

8. The system of claim 1, wherein the server is further connected to a pre-determined remote healthcare system, wherein the pre-determined remote healthcare system transmits the prescription to the server.

9. The system of claim 1, further comprising a restocking module configured to:
detect a quantity of the prescription medication; and
in response to detecting that the quantity of the prescription medication is less than a predetermined quantity, transmit a low inventory signal to the server.

10. The system of claim 1, further comprising a medication clearing module configured to transfer an incorrect prescription medication or an uncollected verified prescribed prescription medication to a compartment, wherein the compartment is inaccessible to public.

11. A method for dispensing a prescription medication, comprising:
identifying, using a user identification module, a user;
establishing a connection between a system for dispensing the prescription medication and a remote server;
retrieving, using a prescription retrieval module, a prescription associated with the identified user from the remote server;
labelling, using a labelling module, the prescribed prescription medication based on the retrieved prescription;
verifying, using a verification module, the labelled prescribed prescription medication, wherein verifying the labelled prescribed prescription medication comprises providing, using an image capturing module, real-time video streaming of the labelling module to a medical personnel; and
dispensing, using a dispensing module, the verified prescribed prescription medication.

12. The method of claim 11, wherein identifying the user comprises capturing a user data for identifying the user.

13. The method of claim 11, wherein labelling the prescribed prescription medication comprises:
printing a label comprising a user name of the user and a medication instruction based on the prescription; and
affixing the printed label to a container containing the prescribed prescription medication.

14. The method of claim 11, further comprising collecting, using a vital signs collection module, one or more vital signs data of the user.

15. The method of claim 14, wherein the vital signs collection module comprises vital signs collection devices, and wherein collecting the one or more vital signs data of the user comprises guiding the user to use the vital signs collection devices via a remote consultation with the medical personnel.

16. The method of claim 14, wherein the one or more vital signs data comprises a body temperature reading, a blood pressure reading, a blood oxygen level reading, a height measurement reading and/or a weight measurement reading.

17. The method of claim 11, further comprising establishing a connection between the remote server and a pre-determined remote healthcare system, wherein the pre-determined remote healthcare system transmits the prescription to the ret-note server.

18. The method of claim 11, further comprising:
detecting, using a restocking module, a quantity of the prescription medication; and
in response to detecting that the quantity of the prescription medication is less than a predetermined quantity, transmitting using the restocking module, a low inventory signal to the remote server.

19. The method of claim 11, further comprising transferring, using a medication clearing module, an incorrect prescription medication or an uncollected verified prescribed prescription medication to a compartment, wherein the compartment is inaccessible to public.

20. The system of claim 4, further comprising a booth in which the vital signs collection module is disposed.
